(19) United States
(12) Patent Application Publication
(10) Pub. No.: US 2025/0009681 A1
(43) Pub. Date: Jan. 9, 2025

(54) SYSTEMS OF ON-DEMAND CUSTOMIZED MEDICAMENT DOSES BY 3D PRINTING

(71) Applicant: Daya Medicals, Inc., Reno, NV (US)

(72) Inventors: Justin DAYA, Reno, NV (US); Kantilal Kasan DAYA, Reno, NV (US)

(21) Appl. No.: 18/887,108

(22) Filed: Sep. 17, 2024

Related U.S. Application Data
(63) Continuation of application No. 18/337,593, filed on Jun. 20, 2023, now abandoned.
Application No. 18/337,593 is a continuation of application No. 17/653,388, filed on Mar. 3, 2022, now abandoned.
Application No. 17/653,388 is a continuation of application No. 15/986,449, filed on May 22, 2018, now abandoned.
Application No. 15/986,449 is a continuation of application No. 15/458,723, filed on Mar. 14, 2017, now abandoned.
Application No. 15/458,723 is a continuation of application No. 13/750,629, filed on Jan. 25, 2013, now abandoned.
(60) Provisional application No. 61/590,994, filed on Jan. 26, 2012.

(51) Int. Cl. A61K 9/70; A61J 3/06; A61K 9/20; B29C 64/20; B29C 64/386; B33Y 30/00; B33Y 80/00

(52) U.S. Cl. CPC A61K 9/70; A61J 3/06; A61K 9/2072; B29C 64/20; B29C 64/386; B33Y 30/00; B33Y 80/00

(57) ABSTRACT

Systems and methods of fabricating a customized dose using a medication 3D printer are provided. According to one embodiment, a system is provided that includes a 3D printer that receives prescription dose instructions from a computer or mobile communication device via a network and assembles a customized medication dose according to the prescription dose instructions by selective application of materials stored in a medicament compound container having at least one reservoir containing the materials for assembly. The materials for assembly can be provided to the 3D printer by a master conduit connecting reservoirs in the medicament compound container to the 3D printer. The 3D printer accesses specified amounts of the materials, which include medicament compounds, via the master conduit, and assembles the customized medication dose according to the prescription dose instructions.

### CROSS-REFERENCE TO RELATED APPLICATIONS

This application is a continuation of U.S. application Ser. No. 18/337,593, filed Jun. 20, 2023, which is a continuation of U.S. application Ser. No. 17/653,388, filed Mar. 3, 2022, now abandoned, which is a continuation of U.S. application Ser. No. 15/986,449, filed May 22, 2018, now abandoned; which is a continuation of U.S. application Ser. No. 15/458,723, filed Mar. 14, 2017, now abandoned, which is a continuation of U.S. application Ser. No. 13/750,629, filed Jan. 25, 2013, now abandoned, which claims priority to U.S. Provisional Application Ser. No. 61/590,994, filed Jan. 26, 2012, which are hereby incorporated by reference in their entirety, including any figures, tables, or drawings, to the extent they are not inconsistent with the teachings explicitly set forth herein.

### BACKGROUND

Additive fabrication technologies, also referred to as direct digital manufacturing, rapid prototyping, and three-dimensional (3D) printing, are becoming increasingly pervasive in manufacturing and design. In general, a 3D prototype formed by an additive fabrication technology is based on what can effectively be considered two-dimensional (2D) layers that form the 3D object. In particular, a digital representation of an object is constructed, for example, via 3D computer-aided design (CAD), and the digital representation can be stored in a computer memory. Computer software may be used to section the representation of the object into a plurality of distinct 2D layers, or “slices” (an x-y cross-section with a nominal and/or technology determined z thickness). Each layer of the 3D prototype is created according to a slice of the CAD drawing. Each slice represents a single layer of the constructed object and the thickness of the slice is based on the resolution of the particular 3D printer or rapid prototyping device used. Together, the fabricated slices form the desired object.

There are a variety of technologies available for 3D printing. Each 3D printing technology has a particular method for building layers to create structures and may use different materials and binding processes. Some methods use melting or softening material to produce the layers. Examples of such methods include selective laser sintering and fused deposition modeling. Other methods lay liquid materials that are then cured using a particular technology. Lasers and photopolymers may be used. An example of a curing method is stereolithography where a laser is used to cure the shape of a 3D CAD model on a platform located in a vat of resin from the bottom up, one layer at a time. Another method of 3D printing uses an inkjet printing system. The printer creates the model one layer at a time by spreading a layer of plaster or resin powder and printing, via an inkjet, a binder onto the layer of powder in the cross-section of the part. The process is repeated until every layer is printed. Regardless of the physical printing process, 3D printing generally begins from CAD drawings that are sliced into layers that act as templates for printing.

The additive fabrication technologies are currently finding use in the footwear, industrial design, architecture, engineering, construction, automotive, aerospace, dental, and medical industries. Medical applications of 3D printing are in early stages and include a wide variety of applications. For example, surgeons are starting to use 3D printers to create practice models for complex surgeries based on images from CT scans. More generic models are being used to explain specific procedures to patients who may have trouble following technical jargon without a visual 3D aid. 3D printing is also beginning to emerge in the prosthetics industry, providing for example artificial bones and masks for patients requiring prosthetic noses or ears. Three dimensional printing technology is also currently being studied by biotechnology firms and academia for possible use in tissue engineering applications where organs and body parts are built using inkjet techniques.

### BRIEF SUMMARY

Systems and methods are provided for assembling a 3D printed customized dose utilizing a medication 3D printing system. According to one embodiment, a 3D printer receives a prescription dose instruction through a network. The prescription dose instruction can specify the color, shape, size, design, pattern, smell, taste, quantity, or any other characteristic of the customized 3D printed dose. Once the prescription dose instructions are received, the 3D printer communicates with a medicament compound container to determine whether the needed compounds/materials are available and to prepare for printing the dose.

In certain embodiments, the medicament compound container can be contained within the 3D printer to form a single unit. In some embodiments, the medicament compound container can be external from the 3D printer. The medicament compound container can include at least one reservoir, each reservoir able to retain a medicament compound, filler material, or other material safe for human consumption.

Medicament compounds can be in solid, liquid, or semi-liquid form and can include active or inactive ingredients, which can be selectively utilized in particular quantities to enable a fine-tune adjustment of strength and dosage. In one embodiment, a single reservoir may contain a mixture of active and inactive ingredients. In addition to medicament compounds, homeopathic, nutraceutical, and herbal compounds and/or materials may be used.

In another aspect of an embodiment of the present invention, a method of treating a patient with a one-a-day, orally administered, treatment regimen is provided, including selecting one of the plurality of treatment regimens and titrating the dosages supplied to the patient after weekly, monthly or quarterly periods of time. Some embodiments of the present invention facilitate the selection of a treatment regimen and/or the change in dosages or titration of dosages of a treatment regimen over time.

According to one embodiment, each reservoir is connected to a conduit and each reservoir's conduit is connected to a master conduit to transport the medicament compounds from the reservoirs in the medicament compound container to the 3D printer. After the medicament compounds have been provided to the 3D printer, a customized 3D printed dose is assembled according to the prescription dose instruction previously provided to the 3D printer.

The medication 3D printing system of some embodiments of the invention provides advantages including the ability to assemble customized 3D printed doses that contain several medicament compounds. In certain embodiments, a customized 3D printed dose may eliminate the need for a patient to take several different pills. Medicament compounds can be utilized to assemble customized 3D printed doses in specific strengths determined by the prescription dose instructions. A predetermined strength or dosage of a particular medicament printed-to-order, as provided in certain embodiments of the invention, can inhibit a patient from overdosing.

In a further embodiment, customized 3D printed pills can be assembled by layering medicament compounds in a fashion to create time-controlled release of medicament compounds.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 shows a medication 3D printer system configuration according to an embodiment of the invention.

FIGS. 2A-2C show representations of different types of medicament compound containers in accordance with certain embodiments of the invention.

### DETAILED DISCLOSURE

Systems and methods are provided for on-demand medication and health-related products. According to certain embodiments, a 3D printed customized dose is assembled utilizing a medication 3D printing system, enabling medicine on-demand.

According to one embodiment, a 3D printer receives a prescription dose instruction through a network. The prescription dose instruction can specify the color, shape, size, design, pattern, smell, taste, quantity, or any other characteristic of the customized 3D printed dose. Once the prescription dose instructions are received, the 3D printer communicates with a medicament compound container to determine whether the needed compounds/materials are available and to prepare for printing the dose. If the 3D printer determines that a compound or material is missing or not available in a needed amount, a message may be sent to the device on which the prescription dose instructions were input and/or to a pharmacy or other location capable of providing a refill or sending a replacement medicament compound container.

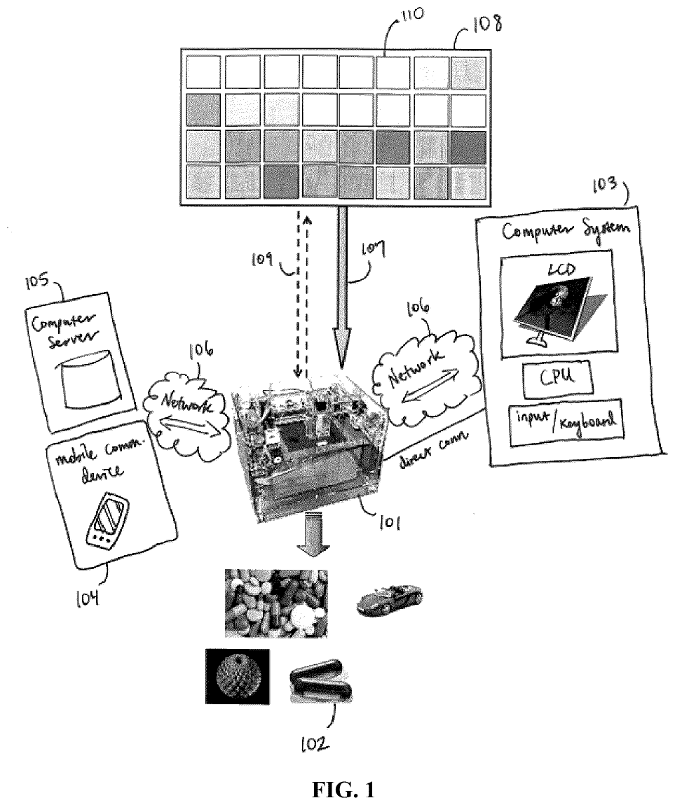

FIG. 1 illustrates an embodiment of the medication 3D printer system. In accordance with an embodiment of the invention, a 3D printer 101 is used to create a 3D printed customized dose 102. The medication can be in the form of a liquid, a pill, or a semi-liquid. Medication, also referred to as a medicament or a medicine, is typically a drug or any other substance used to inhibit or cure disease or to relieve pain or any form of perceived discomfort. In order to facilitate delivery of a particular substance that is used to treat a person, the medicament includes active ingredients (e.g., the pharmacon) and inactive ingredients (e.g., a carrier or excipient). The active ingredients can be in the form of a powder or a liquid and can be mixed with inactive ingredients already designated as safe for human consumption. Although herbal treatments may not have an identified “active” ingredient due to unknown causation and/or required cofactors, known or supposed treatment ingredients can be considered “active” ingredients in accordance with some embodiments of the invention.

According to certain embodiments of the medication 3D printer system, a user can utilize a computer system 103 or a mobile communication device 104 to input the prescription dose information. In one embodiment, a printer driver and application user interface can be provided for facilitating the input of the prescription dose information from the computer system 103 or mobile communication device 104 to the 3D printer. A computer 103 or mobile communication device 104 can provide the 3D printer with a set of instructions that specify the fabrication parameters of the 3D printed customized dose 102.

User input devices can include, but are not limited to a keyboard, touchscreen, or microphone. The parameters contained within the set of instructions can include, but are not limited to, one or more of the following: the dosage of one or more medicament compounds (active and/or inactive) to be included in the 3D printed pill, the volume of the one or more medicament compound; the color of the medicament compound or the color of the 3D printed pill; the shape of the 3D printed pill; a specific design of the 3D printed pill; a pattern, picture, or phrase on the 3D printed pill; the size of the 3D printed pill; the smell or taste of the 3D printed pill; and the quantity of 3D pills printed. For example, in one embodiment, the design elements for the 3D printed pill can be relatively fixed such that a user simply selects a pre-programmed shape/design of the pill and a prescribed dose can be printed into the pre-programmed shape/design. A 3D printed pill for a child can be in the shape of known book, movie, game, or TV show characters.

The computer (or computing device) 103 can be directly connected to the 3D printer 101 or the computer 103 can communicate the fabrication parameters contained within the prescription dose instructions to the 3D printer 101 over a network 106. Communication between elements over the network can be wired or wireless.

The network can be, but is not limited to, a cellular (e.g., wireless phone) network, the Internet, a local area network (LAN), a wide area network (WAN), a WiFi network, or a combination thereof. Such networks are widely used to connect various types of network elements, such as routers, servers, and gateways. It should also be understood that the invention can be practiced in a multi-network environment having various connected public and/or private networks. As will be appreciated by those skilled in the art, communication networks can take several different forms and can use several different communication protocols. Certain embodiments of the invention can be practiced in distributed-computing environments where tasks are performed by remote-processing devices that are linked through a communications network. In a distributed-computing environment, program modules can be located in both local and remote computer-storage media including memory storage devices.

In an embodiment of the invention, a mobile communication device 104 can be used as an alternative or in addition to the computer 103 for wirelessly communicating prescription dose instructions to the 3D printer 101 via the network 106. A mobile communication device 104 can include, but is not limited to, smart phones, tablet computers, and electronic readers. In another embodiment, the prescription dose instructions can be stored on an external computer server 105 and the prescription dose instructions may be retrieved by the 3D printer 101 from the remotely located external computer server 105 through the network.

A medicament compound container 108 is connected to the 3D printer 101 to provide the materials for fabricating the 3D printed pill. In certain embodiments, the 3D printer 101 and medicament compound container 108 can communicate with each other via connection 109. In one embodiment, the medicament compound container 108 communicates with the 3D printer 101 to begin assembling the 3D printed customized dose 102. The communication over connection 109 can involve a sensing circuit providing an output to the 3D printer 101 to determine amounts of available materials. The sensing circuit can also monitor the amount of materials remaining within the medicament compound container 108. A user can set a predetermined level to indicate when the remaining amount of a material or a medicament compound is low. For example, if the low material notification level is set to 25%, when the level of a specific material reaches or falls below 25%, a notification is sent. The notification can contain information about which material or medicament compound is low, the time at which it reached a low level and an estimate of time remaining until the material is fully depleted based on average usage. The notification can be sent to the 3D printer 101 over the connection 109 or a notification can be sent over a network to a computer, computer server or mobile communication device. In one embodiment, security measures can be implemented using the connection 109 to enable fabrication of regulated medications upon verification that a proper use is being conducted.

Medicament compounds can be delivered from the medicament compound container 108 to the 3D printer 101 through a master conduit 107. A reservoir in the medicament compound container 108 can store a medicament compound with a specified concentration. The specified concentration may be a pure form of the medicament compound or it may include fillers, stabilizers, or other compounds enabling the storage of the medicament compound in the reservoir, as well as the form of the compound (e.g., powder, liquid, or other form used by the 3D printer). If the customized dosage requirements for the 3D printed customized dose 102 are less than the specified concentration of the medicament compound, the 3D printer 101 can provide the proper dosage by diluting the specified concentration to a lesser concentration through the addition of an inactive ingredient or filler ingredient. The medicament compound is transferred to the master conduit 107 along with the inactive ingredients and/or filler ingredients and the substances are mixed to obtain the proper customized dosage requirements for the 3D printed customized dose 102.

In certain embodiments, once the 3D printed customized dose 102 is fabricated, the 3D printer 101 executes a clean cycle to remove any residue remaining in the master conduit 107, thereby inhibiting contamination of subsequent doses.

In some embodiments, inactive ingredients and compounds can be combined with natural ingredients to create non-pharmaceutical 3D printed customized doses 102. These non-pharmaceutical 3D printed customized doses can be, but are not limited to, placebo pills, nutraceuticals, herbal supplements, or vitamin supplements. Inactive ingredients and natural ingredients can also be combined with medicament compounds. For example, Chinese herbs, teas, chemicals, and supplements can be combined with medicament compounds.

In an embodiment of the invention, several different medicament compounds, each having a distinct medicinal purpose, can be combined into one 3D printed customized dose 102, thereby reducing the number of pills an individual must take. In another embodiment of the invention, medicament compounds can be layered when the 3D printed customized dose 102 is printed. The layers can include materials that dissolve at particular speeds when ingested. In certain embodiments, the use of these time-release materials may eliminate or reduce the need for an individual to take several pills throughout the day.

In another embodiment of the invention, medicament compounds can be formulated for ingestion by pets and other animals. For example, 3D printed customized doses can be fabricated and used in veterinary practice.

A predetermined strength or dosage of a particular medicament can be printed-to-order, and may be printed at predetermined times. In one embodiment, the printer can communicate with a compliance hub as described in U.S. application Ser. No. 13/091,979, incorporated herein by reference.

The control of when and how often a pill is printed can facilitate compliance as well as reduce misuse. By controlling how often a pill is printed, the subject system can inhibit an individual from overdosing on a prescribed medication, as well as provide additional security over restricted or regulated substances.

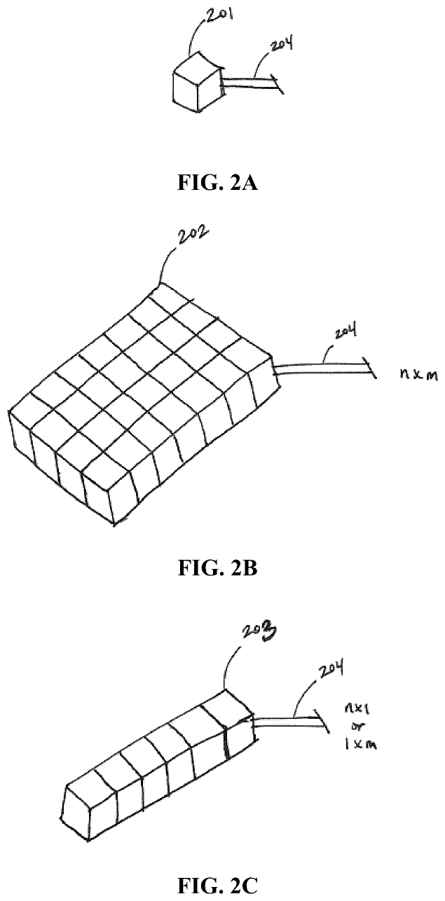

As described above, medicament compounds can be stored in a medicament compound container 108, and the medicament compound container 108 comprises one or more reservoirs 110 in which a solid, liquid, or semi-liquid medicament compound can be kept. FIGS. 2A-2C show example configurations of medicament compound containers in accordance with certain embodiments of the invention. Referring to FIG. 2A, the medicament compound container 108 can comprise a single reservoir 110 as a single medicament compound compartment 201. In certain embodiments using a single compartment, the medicament compound contained in the reservoir may be pre-mixed and the dosage controlled by the size of the printed pill.

The medicament compound container 108 can be modular and configurable into an array or a matrix with N number of rows and M number of columns as shown in FIG. 2B (with n×m compartments 202) and FIG. 2C (with 1×n or 1×m compartments 203)

Each reservoir 110 of the medicament compound container 108 can contain a different medicament compound, and several different reservoirs 110 can contain the same medicament compounds in varying dosages and strengths. In an embodiment of the invention, each reservoir 110 may be attached to the 3D printer 101 through one or more connections 109, thereby enabling communication between the reservoirs 110 within the medicament compound container 108 and the 3D printer 101. In one embodiment, communication can be conducted in series. In another embodiment, communication is conducted in parallel. In certain embodiments, compartments 202, 203 may communicate with each other as well as with the 3D printer.

According to one embodiment, reservoirs 110 in the medicament compound container 108 include a conduit 204. The conduits 204 from each reservoir 110 can be connected to a master conduit 107 that is attached to the 3D printer 101. For example, a single medicament compound compartment 201 with one reservoir 110 may have a single conduit 204 attached to the master conduit 107, and a medicament compound container with 16 compartments 202 (having 16 reservoirs 110) may have up to 16 individual conduits 204 connected to the master conduit 107. The master conduit 107 is used to supply the medicament compounds to the 3D printer 101 from the individual conduits 204 connected to the medicament compound container 108 reservoirs 110.

In an embodiment of the invention, the medicament compound container 108 is externally attached to the 3D printer. In another embodiment of the invention, the medicament compound container 108 is internally contained within the 3D printer.

In an embodiment of the invention, a medical practitioner can enter customized parameters for the fabrication of a 3D printed customized dose 102 based on a medical diagnosis. The 3D printer 101 can be located remotely from a computer system 103 or mobile device 104 used by the medical practitioner to enter the customized parameters. For example, the medical practitioner may provide a medical diagnosis and input customized parameters to the computer system 103 or mobile communication device 104 located at the medical practitioner's office. The information can then be sent wirelessly to the 3D printer 101 which can be located in another room within the medical practitioner's office or hospital, at a retail pharmaceutical store such as WALGREENS or CVS, or even at the residence of the patient. The patient's customized 3D printed dose 102 can be immediately provided or may be picked up from a retail pharmaceutical store (or other location).

In another embodiment of the invention, the consumer can assemble 3D printed customized doses 102 directly through their own personal 3D printer 101. For example, a medical practitioner can provide the individual with a prescription that includes customized parameters for the fabrication of a 3D printed customized dose 102. The individual can then visit or request from a retail pharmaceutical store at least one medicament compound container (or compartment containing a reservoir filled with the medicament compound) according to the prescription. Once the prescription has been filled, the individual can connect the medicament compound container 108 to or within their personal 3D printer to fabricate a 3D printed customized dose 102. The 3D printer 101 can be portable or stationary.

In an embodiment of the invention, the 3D printer 101 is configured to communicate with or incorporate a compliance monitoring device such as the dispensing device described in application Ser. No. 13/091,979, the description of which is hereby incorporated by reference to application Ser. No. 13/091,979. For example, the 3D printer 101 can include a compliance monitoring device or is configured to communicate with a compliance monitoring device that functions to enable an assessment of the degree of a patient's compliance with her therapeutic regimen while the patient is at home, at work, or at any place away from her health care professional's office.

In an embodiment, the Dispensing Module utilizes a dispensing device, such as a Dispensing Device, to capture compliance information regarding a patient's taking of one or more medicaments. In an embodiment, the dispensing device includes sensors adapted to sense the presence, absence, or movement of one or more medicaments and/or their packaging so as to assess whether or not a patient has taken one or more medicaments. In a further embodiment, the dispensing device also includes a communication device adapted to transmit compliance information to one or more persons involved in the care or treatment of the patient. In a particular embodiment, the communication device transmits such compliance information to a server where it is processed and communicated on to one or more persons involved in the care or treatment of the patient.

The dispensing device can be programmed to dispense medicine at a time to the patient according to a health professional's recommendation or prescribed regimen. When it is time for a patient to take a prescribed medicine, the dispensing device can provide a first signal (e.g., a sound) to remind the patient the time for taking medication. According to one embodiment, before dispensing of medicine to a patient, the dispensing device validates the patient's identity using a biometric signature of a patient such as fingerprint, retina, and/or voice. Once the patent is identified as the correct patient, the dispensing device can dispense the medicine out of a slot or aperture of the dispensing device or allow the patient open the dispensing device and take a dispensed medicine (or package or pouch of medicines). The dispensing device may be opened by the patient's touch or voice command For a touch-activated dispensing device, the dispensing device has a touch-sensitive region or mechanically operated fixture that allows the patient (or user) to open the dispensing device. While the dispensing device is touched, the patient's physical information can be obtained in real time. For example, information such as blood pressure, pulse, sugar level, and body temperature can be obtained. For a voice-activated dispensing device, the dispensing device opens upon a predetermined word. The dispensing device can include sensors for determining pitch, duration, and intensity in order to match to the voice-activation word.

A caregiver/administrator may also remotely or non-remotely choose to remove certain medications from the dispensing device, and the dispensing device can then store these selected medications when signaled remotely (wireless) or non-remotely. Therefore, a third party may, over a network or other communication platform, change the order or remove certain individual pouches from the dispensing device or change the order to be dispensed to the patient.

The dispensing device can be programmed to dispense medicine at a time to the patient according to a health professional's recommendation or prescribed regimen. When it is time for a patient to take a prescribed medicine, the dispensing device can provide a first signal (e.g., a sound) to remind the patient the time for taking medication. According to one embodiment, before dispensing of medicine to a patient, the dispensing device validates the patient's identity using a biometric signature of a patient such as fingerprint, retina, and/or voice. Once the patent is identified as the correct patient, the dispensing device can dispense the medicine out of a slot or aperture of the dispensing device or allow the patient open the dispensing device and take a dispensed medicine (or package or pouch of medicines). The dispensing device may be opened by the patient's touch or voice command For a touch-activated dispensing device, the dispensing device has a touch-sensitive region or mechanically operated fixture that allows the patient (or user) to open the dispensing device. While the dispensing device is touched, the patient's physical information can be obtained in real time. For example, information such as blood pressure, pulse, sugar level, and body temperature can be obtained. For a voice-activated dispensing device, the dispensing device opens upon a predetermined word. The dispensing device can include sensors for determining pitch, duration, and intensity in order to match to the voice-activation word.

In an embodiment, the compliance monitoring device includes an application interface for sharing information with other applications. For example, the compliance monitoring device can include an interface for communicating with an electronic medical records (EMR) system. In an embodiment, the memory includes computer-readable media embodying a computer-program product. In an embodiment, the memory includes a database or other device for data storage. Other memory devices are known in the art and can be used with the subject invention.

When medicaments fabricated and dispensed by the 3D printer include coatings or other additive chemicals or structures that when ingested by a patient provide an indication of being in the body of the patient, the external or incorporated compliance monitoring device can receive the compliance data indicating that the medicament was taken by the patient. For example, the compliance monitoring device can communicate to the patient that a breath or blood sample is needed to be taken and when the patient provides the breath or blood sample, the compliance monitoring device can receive the data from a peripheral or internal sensor or device that determines whether an indication of the coating or other chemical or body reaction to the coating or other chemical has occurred. The compliance monitoring device can then communicate such data as needed for determining or reporting compliance.

In addition, an alert with audio and/or visual signals can be provided from the compliance monitoring device to indicate that the patient should access the dispensing portion of the 3D printer to receive her 3D printed customized dose.

In a further embodiment, such compliance information or reports are also accessible to a physician via one or more user devices. In an embodiment, the physician can monitor compliance of some or all of the physician's patients via the compliance management system. In an embodiment, the physician receives alerts of emergent information via mobile phone, PDA, pager, or similar device. In an embodiment, the physician receives summary reports via periodic emails or other messages. Important information can be highlighted in these reports to draw the physician's attention. For example, particularly poor compliance or abnormal blood chemistry results can be presented with colored or flashing text. Other methods of highlighting information are known in the art and can be used with the subject invention. As with a patient, the physician can customize the content, format, timing, and/or delivery mode or the alerts and/or reports. In an embodiment, the physician can also access the compliance monitoring system when needed to obtain information about a particular patient, such as in advance of or during an office visit with the patient. In an embodiment, the compliance management system provides an alternative to a complete EMR system.

In an embodiment, the 3D printer 101 is configured to create a 3D printed customized dose 102 according to a health professional's recommendation or prescribed regimen that can be communicated to the 3D printer. When it is time for a patient to take a prescribed medicine, an external or internally located compliance monitoring device can provide a signal to the 3D printer 101 to begin fabricating the 3D printed customized dose 102.

Communication between an external compliance monitoring device and the 3D printer can be via a wired connection, including, but not limited to, Universal Serial Bus (USB), High Speed Serial Bus (e.g., FIREWIRE), or other electrical connectors (e.g., XLR, Video, plug/socket); or via a wireless connection over a network.

In a further embodiment, once the 3D printer 101 creates a 3D printed customized dose 102, the 3D printed customized dose 102 can be packaged by depositing it into a secure medicament package or medicament pouch that is then stored within a dispenser. The dispenser can hold multiple medicament pouches and each medicament pouch can include one or more 3D printed customized doses 102. A single medicament pouch may also contain a combination of different 3D printed customized doses 102. The combination of 3D printed customized doses 102 in each medicament pouch can be the same or different than that in other medicament pouches in the dispenser.

In an embodiment, the monitoring device comprises at least one sensor capable of sensing information relevant to patient compliance. For example, in an embodiment, the monitoring device includes a barcode scanner and/or radio frequency identification (RFID) reader for scanning identification tags. In a particular embodiment, the at least one sensor senses movement of a MUDP or other medicament packaging as described above. In a particular embodiment, the at least one sensor senses the movement of liquid filled dose packaging, such as for a glaucoma medication. In an embodiment, a sensor can be used to monitor the clinical status of the patient, such as the patient's weight, heart rate, blood pressure, etc. In an embodiment, a sensor obtains the patient's EKG signal. In an embodiment, a sensor can be used to test one or more bodily fluids of the patient, such as blood, urine, sweat, semen, and/or other bodily fluids. For example, the sensor can be used to test one or more aspects of blood chemistry as known in the art. In an embodiment, the sensor provides a complete blood chemistry analysis or profile of the patient. In an embodiment, the sensor measures one or more blood serum levels such as glucose, cholesterol, or other blood serum levels. In an embodiment, a sensor can be used to test a tissue sample from the patient.

In certain embodiments, each medicament pouch or 3D printed customized dose can be dispensed at a predetermined time. For example, the first medicament pouch or 3D printed customized dose can be scheduled to dispense at 8:00 am, the second medicament pouch or 3D printed customized dose can be scheduled to dispense at 12:00 pm, and the third medicament pouch or 3D customized dose can be scheduled to dispense at 4:00 pm. The scheduled dispensing arrangement can be programmed to repeat each day until an ending date determined by a health professional according to a patient's health condition. In the event that a patient misses a dose, the medicament pouch can be retained for disposal or consumption at a later time.

In another embodiment of the invention, 3D printers 101 are located at local businesses that receive high volumes of consumers visiting every day such as grocery stores, gas stations, pharmacies, or superstores. These printers can include medicament compound containers containing common or popular medicament compounds, enabling an individual to obtain their prescription. Alternatively (or in addition), the printers located at the local businesses can include a compartment enabling insertion of a medicament compound container received by an individual from a pharmacy. An individual can then utilize their customized prescription by placing their medicament compound container 108 inter the printer's compartment to create a 3D printed customized dose 102.

Security features can be implemented to protect the safety and well-being of the individuals consuming the 3D printed customized doses 102. In an embodiment of the invention, the 3D printer 101 is activated by authorized computer readable instructions that specify the customized fabrication parameters, thereby inhibiting unauthorized doses or controlled substances from being manufactured and distributed. For prescriptions, a unique password or key code can be provided to permit the execution of pill printing. In another embodiment of the invention, the reservoirs 110 are tamper proof to inhibit contamination of the medicament compounds. The reservoir can contain a transmitter that emits a signal to indicate that the reservoir has been tampered with.

Certain techniques set forth herein may be described in the general context of computer-executable instructions, such as program modules, executed by one or more computers or other devices. Certain embodiments of the invention contemplate the use of a computer system within which a set of instructions, when executed, can cause the 3D printing system to perform any one or more of the methodologies discussed above. Generally, program modules include routines, programs, objects, components, and data structures that perform particular tasks or implement particular abstract data types. In various embodiments, the functionality of the program modules may be combined or distributed as desired over a computing system or environment. Those skilled in the art will appreciate that the techniques described herein may be suitable for use with other general purpose and specialized purpose computing environments and configurations. Examples of computing systems, environments, and/or configurations include, but are not limited to, personal computers, server computers, hand-held or laptop devices, multiprocessor systems, microprocessor-based systems, programmable consumer electronics, and distributed computing environments that include any of the above systems or devices.

In certain embodiments, the 3D printing is employed in a computing environment including a general-purpose computing system in the form of a computer, or a mobile communication or tablet device. A mobile communication device can include broadly any portable electronic device that provides voice, video, or data communication. The computer may include one or more processors or processing units, memory, and system bus for facilitating communications between system components including the processors and memory. A monitor or other display device can be connected to the system bus via an interface. The computer can also include a variety of input devices for enabling a user to enter commands and information into the computing system. In addition, the system bus can enable communication between the 3D printer and the processor(s) and memory. Removable and non-removable computer readable media can be provided.

It should be appreciated by those skilled in the art that computer readable media includes removable and non-removable structures/devices that can be used for storage of information, such as computer readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); or other media capable of storing computer-readable media now known or later developed. Computer readable media should not be construed or interpreted to include any propagating signals.

It should be understood that the examples and embodiments described herein are for illustrative purposes only and that various modifications or changes in light thereof will be suggested to persons skilled in the art and are to be included within the spirit and purview of this application.

We claim:

1. A medication 3D printer system, comprising:
a 3D printer,
a medicament compound container including at least one reservoir, wherein the least one reservoir is connected to a conduit to transport a medicament compound or filler material contained therein to the 3D printer,
wherein the 3D printer comprises an interface configured to receive a prescription dose information over a network and, upon receipt of the prescription dose instruction, the 3D printer formulates a customized dose according to the prescription dose instruction and assembles a customized dose by selective application of the medicament compound and filler material transported over the conduit from the medicament compound container to the 3D printer and the customized dose is provided with a barcode label; and
wherein the 3D printer is configured to receive information from a compliance monitoring system,
the compliance monitoring system being configured to alert a physician, caregiver, and/or other third party healthcare provider, scan the barcode of the customized dose and/or scan a pattern, picture, or phrase printed on the customized dose, receive information from one or more biometric sensors, and communicate with a database,
the compliance monitoring system being configured to transmit medication adherence data based on the barcode scan, scan of the pattern, picture, or phrase printed on the customized dose, or movement of one or more medicament and/or their packaging to the physician, caregiver, or other third party healthcare provider, and to change or titrate a dosage of the customized dose or series of doses printed from the 3D printer in real-time, and
the compliance monitoring system being configured to provide a notification to a pharmacy to send new medications or other formulations or dosages as refills or as additional medications to be loaded into the 3D printer.

2. The medication 3D printer system of claim 1, wherein the 3D printer receives the prescription dose instructions over a network.

3. The medication 3D printer system of claim 1, further comprising a signal connector to enable communication between the medicament compound container and the 3D printer.

4. The medication 3D printer system of claim 3, further comprising a sensor for detecting an amount of the medicament compound or the filler material contained in the medicament compound container, the 3D printer receiving input from the sensor via the signal connector.

5. The medication 3D printer system of claim 1, wherein the medicament compound container is separate from the 3D printer.

6. The medication 3D printer system of claim 1, wherein the medicament compound container is contained within the 3D printer.

7. The medication 3D printer system of claim 1, wherein the prescription dose instructions specify fabrication parameters of the customized dose including at least one parameter selected from the group consisting of a dosage of one or more medicament compounds, a volume of the one or more medicament compounds, a color for the customized dose, a shape of the customized dose, a specific design of the customized dose, a size of the customized dose, a smell of the customized dose, a taste of the customized dose, and a quantity of the customized dose to be assembled.

8. A medication 3D printer system, comprising:
a 3D printer,
a medicament compound container including a plurality of reservoirs, wherein the plurality of reservoirs are connected to a conduit to transport a medicament compound or filler material contained therein to the 3D printer;
wherein the 3D printer comprises an interface configured to receive a prescription dose information over a network and, upon receipt of the prescription dose instruction, the 3D printer formulates a customized dose according to the prescription dose instruction and assembles a customized dose by selective application of the medicament compound and filler material transported over the conduit from the medicament compound container to the 3D printer;
wherein the network is a cellular network, the Internet, a local area network, or a wide area network;
wherein the customized dose is provided with a barcode label; and
wherein the 3D printer is configured to receive information from a compliance monitoring system,
the compliance monitoring system being configured to alert a physician, caregiver, and/or other third party healthcare provider, scan the barcode of the customized dose and/or scan a pattern, picture, or phrase printed on the customized dose, receive information from one or more biometric sensors, and communicate with a database,
the compliance monitoring system being configured to transmit medication adherence data based on the barcode scan, scan of the pattern, picture, or phrase printed on the customized dose, or movement of one or more medicament and/or their packaging to the physician, caregiver, or other third party healthcare provider, and to change or titrate a dosage of the customized dose or series of doses printed from the 3D printer in real-time, and
the compliance monitoring system being configured to provide a notification to a pharmacy to send new medications or other formulations or dosages as refills or as additional medications to be loaded into the 3D printer.

9. The medication 3D printer system of claim 8, wherein the network is a cellular network.

10. The medication 3D printer system of claim 8, wherein the network the Internet.

11. The medication 3D printer system of claim 8, further comprising a signal connector to enable communication between the medicament compound container and the 3D printer.

12. The medication 3D printer system of claim 11, further comprising a sensor for detecting an amount of the medicament compound or the filler material contained in the medicament compound container, the 3D printer receiving input from the sensor via the signal connector.

13. The medication 3D printer system of claim 8, wherein the medicament compound container is separate from the 3D printer.

14. The medication 3D printer system of claim 8, wherein the medicament compound container is contained within the 3D printer.

15. The medication 3D printer system of claim 1, wherein input from the compliance monitoring device to the 3D printer opens the 3D printer containing the customized dose.

16. The medication 3D printer system of claim 1, wherein the one or more biometric sensors are configured to obtain blood pressure, pulse, blood sugar level, and/or body temperature.

17. The medication 3D printer system of claim 1, wherein the physician and/or healthcare provider adjusts the customized dose of the medication produced by the 3D printer remotely.

18. The medication 3D printer system of claim 1, wherein the database stores barcodes of customized doses; pictures, patterns, or phrases printed on the customized dose; biometric sensor information; and/or healthcare provider information.

19. The medication 3D printer system of claim 8, wherein input from the compliance monitoring device opens the 3D printer containing the customized dose.

20. The medication 3D printer system of claim 8, wherein the one or more biometric sensors are configured to obtain blood pressure, pulse, blood sugar level, and/or body temperature.

21. The medication 3D printer system of claim 8, wherein the physician and/or healthcare provider adjusts the customized dose of the medication produced by the 3D printer remotely.

22. The medication 3D printer system of claim 8, wherein the database stores barcodes of customized doses; pictures, patterns, or phrases printed on the customized dose; biometric sensor information; and/or healthcare provider information.
